(19) United States
(12) Patent Application Publication
(10) Pub. No.: US 2011/0190747 A1
(43) Pub. Date: Aug. 4, 2011

(54) DISPOSABLE LED/LASER CATHETER

(75) Inventors: J. Todd DERBIN, Solebury, PA (US); Dymphna DONAGHY, Letterkenny (IE); David O'FLYNN, Bray (IE); Yvonne WOODS, Leitrim (IE)

(73) Assignee: Arista Therapeutics, Inc., Solebury, PA (US)

(21) Appl. No.: 12/697,140

(22) Filed: Jan. 29, 2010

(51) Int. Cl. A61B 18/20

(52) U.S. Cl. 606/14

(57) ABSTRACT

A device is provided for illuminating a tissue surface to be treated. The device includes a catheter, a light emitting diode in the distal tip of the catheter, and a distal cap disposed over the distal end of the catheter. The device includes a handle connected to the proximal end of the catheter. An external source of energy is provided for supplying energy to the light emitting diode and for controlling the light emitting diode.

### BACKGROUND OF THE INVENTION

This invention relates to medical devices and more specifically to such devices for the treatment of body cavities and damaged vessels using electromagnetic energy.

An endoscope with a removable eyepiece is known that includes a sterilizable catheter having a central coherent fiber bundle for carrying an image to a viewing means. An optical lens is provided at the distal end of the optical bundle to focus an image on the distal end of the optical bundle for transmission through the optical bundle. An optical catheter is also known that includes a sterilizable catheter having a central coherent fiber bundle that can be used to carry an image to a viewing means, or that can be used to kill cancer cells. A fluorescent dye can be attached to the cancer cells, and the treated cancer cells can be subsequently exposed to an exciting laser light frequency of 630 nm to kill the cancer cells.

Application of electromagnetic energy to a tissue surface also has been used to heal tissues in several medical treatments. For example, it is known to apply light to a tissue surface in order to heal a pathological state, to remove a stenosis in a blood vessel or for laser welding of tissues, for example in order to treat a rupture in a vessel wall or to perform an anastomosis of two blood vessels. It is also known to use application of electromagnetic energy for tissue regeneration and therapy. For example, low level laser irradiation (LLLI) in the visible to infrared range of the light spectrum has been clinically shown to accelerate healing in skin wounds, and reduce pain and inflammation in musculoskeletal disorders. The underlying mechanisms are initiating (biostimulating) processes such as collagen synthesis, cell proliferation, and reducing secretion of inflammatory markers. Gavish et al., Lasers in Surgery and Medicine (2006) 38:779-786, which is incorporated herein by reference, discloses that low level laser in vitro stimulates vascular smooth muscle cell proliferation and collagen synthesis, modulates the equilibrium between regulatory matrix remodeling enzymes, and inhibits pro-inflammatory IL-1-β gene expression.

An apparatus has been described for applying electromagnetic energy to the heart tissue for a biostimulative and cytoprotective effect. An apparatus to provide electromagnetic biostimulation of tissue, which includes a source of electromagnetic radiation and optics operatively connected to the source of electromagnetic radiation, can be used for directing electromagnetic radiation from the outside of the body to the tissue surface.

Additionally, low energy light exposure has been found to both inhibit restenosis following dilation of a stenotic region, and to inhibit vascular spasms, whether or not they are associated with a stenotic region. Such light energy has also been found to arrest progress of various types of a stenosis and expose a vessel wall to light energy from an intravascular approach for the prevention of restenosis.

It is also known that an apparatus may be used for applying light to the interior surface of a vascular wall for laser treatment of the vessel. Light may be generated by an extracorporeal light source guided by a light guide to the interior of the blood vessel to be treated. A light deflector and diffuser may be used to direct the light in a substantially radial fashion onto the vessel wall.

Abdominal Aortic Aneurysm (AAA) formation is an arteriosclerotic process characterized by marked disruption of the musculoelastic lamellar structure of the media. Rupture of an untreated AAA is particularly life threatening. Extensive destruction of the elastic tissue is associated with marked inflammatory cell infiltration and progressive diminution in the number of viable smooth muscle cells. Over time, and aggravated by contributory risk factors such as systolic hypertension, aneurysm growth occurs through a complicated, but insidious, imbalance between matrix protein production and degradation, favoring expansion, thereby increasing the risk of rupture of the weakened wall.

AAA is present in approximately 10% of individuals over the age of 65 years, with its frequency increasing as the proportion of elderly individuals in the general population continues to rise. It is widely known that the risk of rupture increases in approximate proportion to aneurysm size, which can be monitored by computed tomography (CT), ultrasound, or magnetic resonance imaging (MRI). The estimated risk of rupture ranges from 10-20% for an abdominal aneurysm 6-7 cm in diameter, to 30-50% if the maximum diameter is greater than 8 cm. Overall mortality from a ruptured AAA is greater than 90%. Current forms of aneurysm treatment focus either on the open abdomen, surgical, graft-based repair or endovascular exclusion of the diseased segment of aorta with large, membrane-covered, e.g. Gortex covered stents. Both techniques have major side effects with potentially life-threatening consequences, particularly in patients of advanced age (the majority of patients) or others at high risk or compromised cardiac function.

Gertz et al. WO 2007/113834, which is incorporated herein by reference, discloses a device and method for illuminating a tissue surface. In Gertz, a light source is optically coupled to the proximal end of a light guide and a light scatterer is optically coupled to the distal end of the light guide. The device includes a deployment mechanism that is configured to bring the light scatterer from an undeployed small caliber configuration in which the light scatterer is delivered to the body surface to a deployed large caliber configuration in which the light scatterer irradiates the body surface.

While the techniques described in the above references describe generally the benefits of the techniques and methods for using the electromagnetic spectrum to treat tissue surfaces, the use of a remote light source and optical paths can create difficulties in the in vitro use of the processes and apparatus in the prior art, such as localized heating, and are not intended to be placed for long periods of time to vary the form and energy of treatment of the tissue. Accordingly, it would be desirable to provide an implantable biocompatible apparatus for the treatment of an interior surface of a damaged vessel or internal body cavity by electromagnetic energy for extended periods of time. The present invention meets these and other needs.

### SUMMARY OF THE INVENTION

Briefly, and in general terms, the present invention provides for a disposable light emitting diode (LED)/laser catheter for low level laser irradiation (LLLI). The catheter of the device can be delivered laparoscopically to position the distal functional tip adjacent to a tissue surface to be treated, and is capable of being left in place for up to 18 hours. The device thus is capable of delivering low-level laser irradiation laparoscopically to internal sites within the body, and in particular is capable of delivering low-level laser irradiation laparoscopically to an abdominal aortic aneurysm. The device is capable of delivering the required dose multiple times at intervals and for durations determined by the patient illness. The device is designed to be removed and disposed of after it is removed. It is a single use sterile device. The device has a separate power source to allow the generation of the energy to be delivered.

Accordingly, the present invention provides for a device for illuminating a tissue surface to be treated, including a catheter having a proximal end and a distal end, the catheter including an interior channel extending between the proximal and distal ends of the catheter, light emitter means for emitting light from the distal end of the catheter, and a distal cap disposed over the distal end of the catheter configured to transmit the light distally for illuminating the tissue surface to be treated. In a presently preferred aspect, the device includes a handle having a proximal end and a distal end.

In a presently preferred aspect, the light emitter means for emitting light includes a light emitting diode disposed in the distal end of the catheter, and in a presently preferred aspect of the third embodiment, an external source of energy is operatively connected to the light emitting diode for supplying energy to the light emitting diode and for controlling the light emitting diode. In another presently preferred aspect, the light emitting diode is a laser diode. In another presently preferred aspect, the distal cap is disposed over the distal end of the catheter and the distal end of the light emitting diode.

These and other aspects and advantages of the invention will become apparent from the following detailed description and the accompanying drawings, which illustrate by way of example the features of the invention.

### BRIEF DESCRIPTION OF THE DRAWINGS

FIG. 1 is a perspective view of the first embodiment of the device for illuminating a tissue surface to be treated, according to the present invention.

FIG. 2 is a top plan view of the device of FIG. 1.

FIG. 3 is a cross-sectional view of the device of FIG. 1, taken along lines 3-3 of FIG. 2.

FIG. 4 is an enlarged view of the distal tip of the device of FIG. 3.

### DETAILED DESCRIPTION OF THE PREFERRED EMBODIMENTS

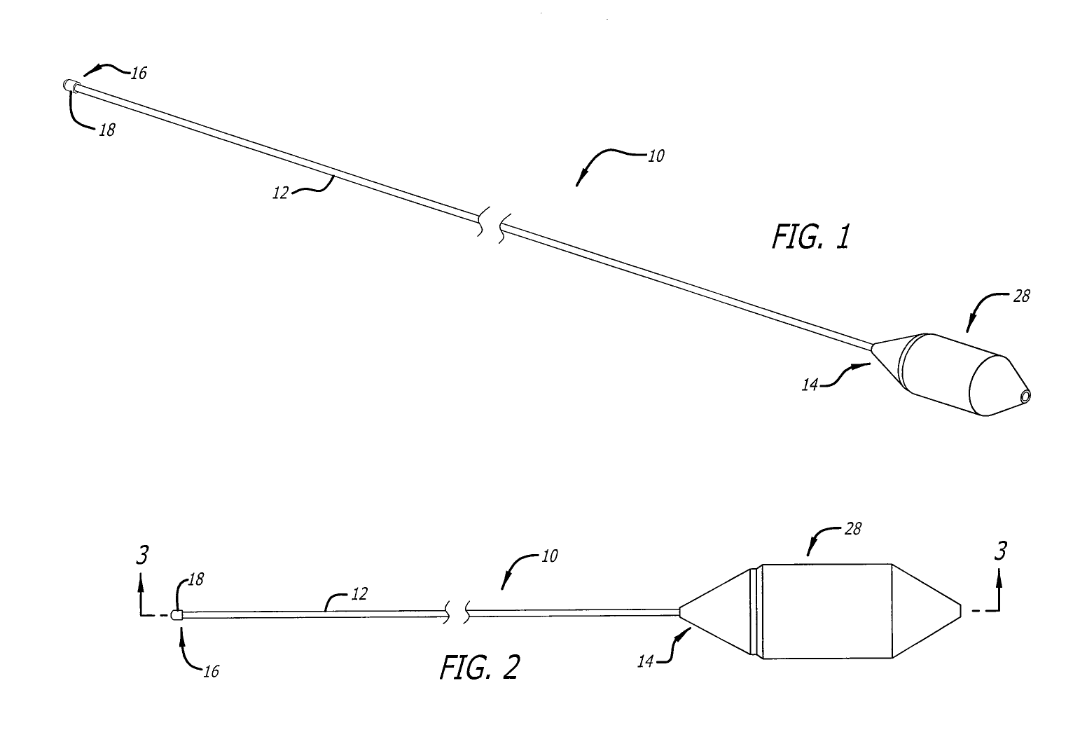

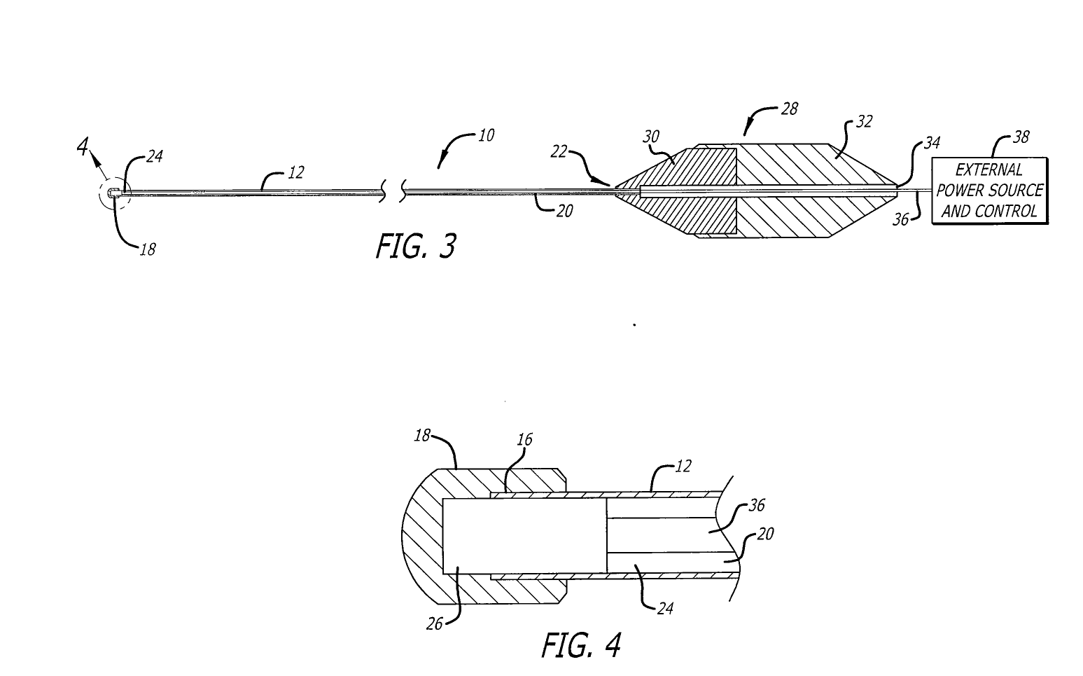

Referring to the drawings, which are provided by way of example, and not by way of limitation, in a first embodiment, the present invention provides for a device for illuminating a tissue surface to be treated, such as a disposable LED/laser catheter for low level laser irradiation (LLLI) 10. Referring to FIGS. 1 and 2, the device includes a catheter 12, which is typically a plastic single lumen catheter, having a proximal end 14 and a distal end 16, and a distal lens or cap 18 mounted over the distal end of the catheter. Referring to FIGS. 3 and 4, the catheter includes a catheter channel 20 extending approximately the length of the catheter, the catheter channel having a proximal end 22 and a distal end 24. The device includes a functional tip, including a light emitting diode (LED) 26 disposed in the distal end of the catheter channel. The light emitting diode is preferably a laser diode capable of delivering a spectrum of light having a wavelength in the range of approximately 700-900 nm, and more preferably light having a wavelength of approximately 780 nm, and is capable of delivering a range of doses from 4 mW/cm2 upwards for a desired period of time to deliver a desired total energy density. Referring to FIG. 4, the distal lens or cap is preferably bonded over the distal end of the catheter and the light guide, and is configured to transmit light from the light guide distally for illuminating the tissue surface to be treated.

The device also typically has a proximal handle 28 including an adapter portion 30 of the handle connected to the proximal end of the catheter, and an end cap 32 connected to the adapter portion at the proximal end of the handle. The handle also includes an interior handle channel 34 extending through the adapter portion and end cap portion of the handle, for electrical wiring 36 extending through the handle channel and the catheter channel and connecting the LED with an external power source and control 38 for controlling operation of the device.

It will be apparent from the foregoing that while particular forms of the invention have been illustrated and described, various modifications can be made without departing from the spirit and scope of the invention. Accordingly, it is not intended that the invention be limited, except as by the appended claims.

We claim:

1. A device for illuminating a tissue surface to be treated, comprising:
a catheter having a proximal end and a distal end, the catheter including an interior channel extending between said proximal and distal ends of said catheter;
light emitter means for emitting light from said distal end of said catheter; and
a distal cap disposed over said distal end of said catheter configured to transmit said light distally for illuminating the tissue surface to be treated.

2. The device of claim 1, further comprising a handle having a proximal end and a distal end, said handle being connected to said proximal end of said catheter.

3. The device of claim 1, wherein said light emitter means for emitting light comprises a light emitting diode disposed in said distal end of said catheter.

4. The device of claim 3, wherein said light emitting diode comprises a laser diode.

5. The device of claim 1, wherein said light emitter means for emitting light comprises a light emitting diode disposed in said distal end of said catheter, and an external source of energy operatively connected to said light emitting diode for supplying energy to said light emitting diode and for controlling said light emitting diode.

6. The device of claim 5, wherein the distal cap is disposed over the distal end of the catheter and the distal end of the light emitting diode.

7. The device of claim 6, wherein said light emitting diode is a laser diode.

8. The device of claim 6, wherein said light emitting diode is operative to provide a spectrum of light having a wavelength in the range of approximately 700-900 nm.

9. The device of claim 8, wherein said light emitting diode is operative to provide a spectrum of light in a dose equal to or greater than 4 mW/cm2.

10. A device for illuminating a tissue surface to be treated, comprising:
a catheter having a proximal end and a distal end, the catheter including an interior channel extending between said proximal and distal ends of said catheter;
a light emitting diode disposed in said distal end of said catheter for emitting light from said distal end of said catheter; and
a distal cap disposed over said distal end of said catheter configured to transmit said light distally for illuminating the tissue surface to be treated.

11. The device of claim 10, further comprising a handle having a proximal end and a distal end, said handle being connected to said proximal end of said catheter.

12. The device of claim 10, wherein the distal cap is disposed over the distal end of the catheter and the distal end of the light emitting diode.

13. The device of claim 10, wherein said light emitting diode is a laser diode.

14. The device of claim 10, further comprising an external source of energy operatively connected to said light emitting diode for supplying energy to said light emitting diode and for controlling said light emitting diode.

15. The device of claim 10, wherein said light emitting diode is operative to provide a spectrum of light having a wavelength in the range of approximately 700-900 nm.

16. The device of claim 10, wherein said light emitting diode is operative to provide a spectrum of light in a dose equal to or greater than 4 mW/cm2.
